(19) United States
(12) Patent Application Publication
(10) Pub. No.: US 2012/0322768 A1
(43) Pub. Date: Dec. 20, 2012

(54) COMPOUNDS, COMPOSITIONS AND METHODS FOR TREATING PROTOZOAN INFECTIONS

(75) Inventors: Longqin Hu, Belle Mead, NJ (US); Shane R. Wilkinson, Billericay (GB); Xinghua Wu, Highland Park, NJ (US); Belinda S. Hall, Kingston Upon Thames (GB)

(73) Assignees: QUEEN MARY & WESTFIELD COLLEGE, London (GB); RUTGERS, THE STATE UNIVERSITY OF NEW JERSEY, New Brunswick, NJ (US)

(21) Appl. No.: 13/511,560

(22) PCT Filed: Nov. 24, 2010
(86) PCT No.: PCT/US2010/058043

§ 371 Date: Aug. 17, 2012

Related U.S. Application Data
(60) Provisional application No. 61/281,872, filed on Nov. 24, 2009.

(51) Int. Cl. A61K 31/664; A61K 31/675; A61P 33/02; C07F 9/24

(52) U.S. Cl. 514/110; 558/166; 514/118

(57) ABSTRACT

Provided are compounds, compositions and methods for treating protozoan infections.

### CROSS REFERENCE TO RELATED APPLICATIONS

This application claims priority from U.S. Provisional Patent Application No. 61/281,872, filed on Nov. 24, 2009, which is incorporated herein by reference in its entirety. This application is also related to US Patent Publication No. US-2004/0214798, entitled “Nitroaryl Phosphoramide Compositions And Methods For Targeting And Inhibiting Undesirable Cell Growth Or Proliferation”, by Hu.

### FIELD OF THE INVENTION

This invention generally relates to compounds, compositions and methods for treatment of protozoan infections, and more particularly, to compounds, compositions and methods for treatment of certain protozoan infections by administration of a nitrobenzyl phosphoramide mustard.

### BACKGROUND OF THE INVENTION

Protozoan infections are responsible for more than 60,000 deaths per year. Over 10 million people are infected by the parasites Trypanosoma brucei and Trypanosoma cruzi, the causative agents of human African trypanosomasis (HAT) and Chagas disease, respectively. The primary route of transmission for both parasites is by the blood-sucking feeding habits of insect vectors. However, other important pathways have been reported, notably blood transfusion, organ transplantation and illicit drug usage. Infections by these alternative routes have become a problem in the developed world.

Other protozoan infections include, but are not limited to, human infective West and East African trypanosomasis caused by Trypanosoma brucei gambiense and Trypanosoma brucei rhodesiense respectively; Hagana caused by Trypanosoma brucei brucei in cattle (used as a model organism for the human African trypanosomasis); Nagana caused by Trypanosoma congolense in cattle and other animals including sheep, pigs, goats, horses and camels; Nagana caused by Trypanosoma vivax in cattle; human infective Chagas disease caused by Trypanosoma cruzi; a spectrum of infections caused by numerous different species (21 species infect humans) of Leishmania including cutaneous leishmnaiasis, visceral leishmnaiasis and mucocutaneous leishmnaiasis; and diarrhea, dysentery, and vaginitis caused by Giardia, Entamoeba, and Trichomonas, respectively.

With no prospect of a vaccine, drugs are currently the only viable option to treat these pathogens. One of the primary drugs presently used to treat protozoan infections is nifurtimox. Treatment with nifurtimox, however, involves several complications. Nifurtimox is most effective in the acute early stage of infection. The benefit of nifurtimox treatment diminishes as the parasitic infection progresses to the chronic stage. Also, nifurtimox has been reported as having several serious side effects, including: gastrointestinal disturbances, headache, vertigo, central nervous system toxicity including disorientation, disturbances of equilibrium such as ataxia, nystagmus, forgetfulness, insomnia, irritability, phychosis, seizures, tremors, eosinophilia, impotence, leukopenia, muscle weakness, and peripheral neurophathy.

Accordingly, there exists a need for novel methods of treatment for protozoan infections that utilize alternate compounds or compositions having increased efficacy and reduced side effects in comparison to currently-used drugs such as nifurtimox.

### SUMMARY OF THE INVENTION

An aspect of the present invention provides compounds, compositions and methods for treating protozoan infections caused by Trypanosoma brucei brucei, Trypanosoma brucei rhodesiense, Trypanosoma brucei gambiense, Trypanosoma congolense, Trypanosoma vivax, Trypanosoma cruzi, Leishmania species, Giardia, Entamoeba, Trichomonas, and/or an additional organism expressing Type I NTR by administration of a nitrobenzyl phosphoramide mustard.

Another aspect of the present invention provides methods using a pharmaceutical composition comprising a pharmaceutically acceptable excipient and a therapeutically effective amount of a nitrobenzyl phosphoramide mustard to treat an infection caused by Trypanosoma brucei brucei, Trypanosoma brucei rhodesiense, Trypanosoma brucei gambiense, Trypanosoma congolense, Trypanosoma vivax, Trypanosoma cruzi, Leishmania species, Giardia, Entamoeba, Trichomonas, and/or an additional organism expressing Type I NTR.

Another aspect of the present invention provides a method for treating a protozoan infection comprising administering an effective amount of nitrobenzyl phosphoramide mustard to a patient in need thereof.

### BRIEF DESCRIPTION OF THE DRAWINGS

FIG. 1 depicts a proposed mechanism for the activation of nitrobenzyl phosphoramide mustards, in accordance with an embodiment of the present invention;

FIG. 2 depicts the activity of TbNTR toward different nitrobenzyl phosphoramide mustards, in accordance with an embodiment of the present invention;

FIG. 3 depicts the susceptibility of bloodstream form TT brucei with altered levels of NTR to nitrobenzyl phosphoramide mustards, in accordance with an embodiment of the present invention; and

FIG. 4 depicts a stability study of two active compounds used in an embodiment of the present invention in phosphate buffer and in whole human blood.

### DETAILED DESCRIPTION

The invention disclosed herein is intended to encompass compounds, compositions and methods employing all pharmaceutically acceptable salts thereof of the disclosed nitrobenzyl phosphoramide mustards. The pharmaceutically acceptable salts include, but are not limited to, metal salts such as, e.g., sodium salt, potassium salt, cesium salt and the like; alkaline earth metals such as, e.g., calcium salt, magnesium salt and the like; organic amine salts such as, e.g., triethyline salt, pyridine salt, picoline salt, ethanolamine salt, triethanolamine salt, dicyclohexylamine salt, N,N′-dibenzylethylenediamine salt and the like; inorganic acid salts, such as, e.g., hydrochloride, hydrobromide, sulfate, phosphate and the like; organic acid salts, such as, e.g., formate, acetate, trifluoroacetate, maleate, fumarate, tartrate and the like; sulfonates, such as, e.g., methanesulfonate, benzenesulfonate, p-toluenesulfonate and the like; amino acid salts, such as, e.g., arginate, asparginate, glutamate and the like.

The invention disclosed herein is also intended to encompass methods employing all prodrugs of the disclosed nitrobenzyl phosphoramide mustards. Prodrugs are considered to be any covalently bonded carriers which release the active parent drug in vivo. An example of a prodrug would be an ester which is processed in vivo to a carboxylic acid or salt thereof.

The invention disclosed herein is also intended to encompass the in vivo metabolic products of the disclosed nitrobenzyl phosphoramide mustards. Such products may result for example from the oxidation, reduction, hydrolysis, amidation esterification and the like of the administered compound, primarily due to enzymatic processes. Accordingly, the invention includes compounds produced by a process comprising contacting a compound of this invention with a mammal for a period of time sufficient to yield a metabolic product thereof. Such products typically are identified by preparing a radio-labeled compound of the invention, administering it parenterally in a detectable dose to an animal such as, e.g., a rat, mouse, guinea pig, monkey, or human, allowing sufficient time for metabolism to occur and isolating its conversion products from the urine, blood or other biological samples. One skilled in the art recognizes that interspecies pharmacokinetic scaling can be used to study the underlining similarities and differences in drug disposition between species, to predict drug disposition in an untested species, to define pharmacokinetic equivalence in various species, and to design dosage regimens for experimental animal models, as discussed in Mammals, 1028, Journal of Pharmaceutical Sciences, Vol. 75, No. 11, November 1986.

The invention disclosed herein is also intended too encompass the disclosed notrobenzyl phosphoramide mustards being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. Some of the compounds disclosed herein may contain one or more asymmetric centers and may thus give rise to enantiomers diastereomers, and other stereoisomeric forms. The present invention is also intended to encompass all such possible forms as well as their racemic and resolved forms and mixtures thereof. When the compounds described herein contain olefinic double bonds or other centers of geometric asymmetry, and unless specified otherwise, it is intended to include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “enantiomer” or “enantiometric” refers to a molecule that is non-superimposable on its minor image and hence optically active wherein the enantiomer rotates the plane of polarized light in one direction and its mirror image rotates the plane of polarized light in the opposite direction.

The term “racemic” refers to a mixture of equal parts of enantiomers and which is optically active.

The term “resolution” refers to the separation or concentration or depletion of one of the two enantiomeric forms of a molecule.

An embodiment of the present invention advantageously provides methods of treatment for protozoan infections that utilize alternate compounds or compositions having increased efficacy and reduced side effects in comparison to currently-used drugs such as nifurtimox.

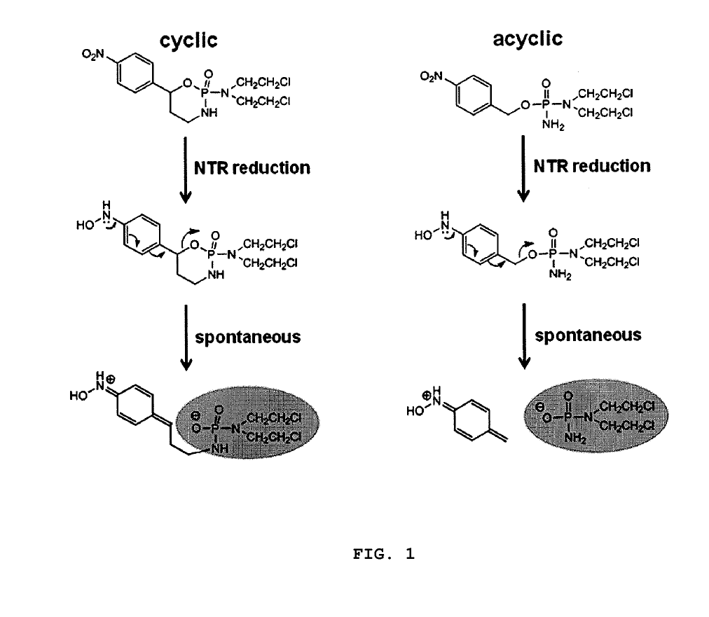

FIG. 1 depicts a proposed mechanism for the activation of nitrobenzyl phosphoramide mustards. Two types of NBPM are shown. One form contains the mustard as part of a cyclic arrangement and is analogous to cyclophosphamide. The nitroreductase-mediated reduction of the nitro group (electron withdrawing) to hydroxylamine (electron-donating) causes a rearrangement of electrons within the NBPM backbone. This promotes the cleavage of a C—O bond, activating a cytotoxic phosphoramide mustard moiety (shaded). It is proposed that this molecule triggers DNA damage by acting as an alkylating agent (15, 16). After nitro-reduction the cyclophosphamide structure is predicted to undergo ring opening exposing the cytotoxic mustard. In the second form, the mustard is part of a linear (acyclic) structure. Here, the NBPM is postulated to fragment, releasing the cytotoxic phosphoramide mustard.

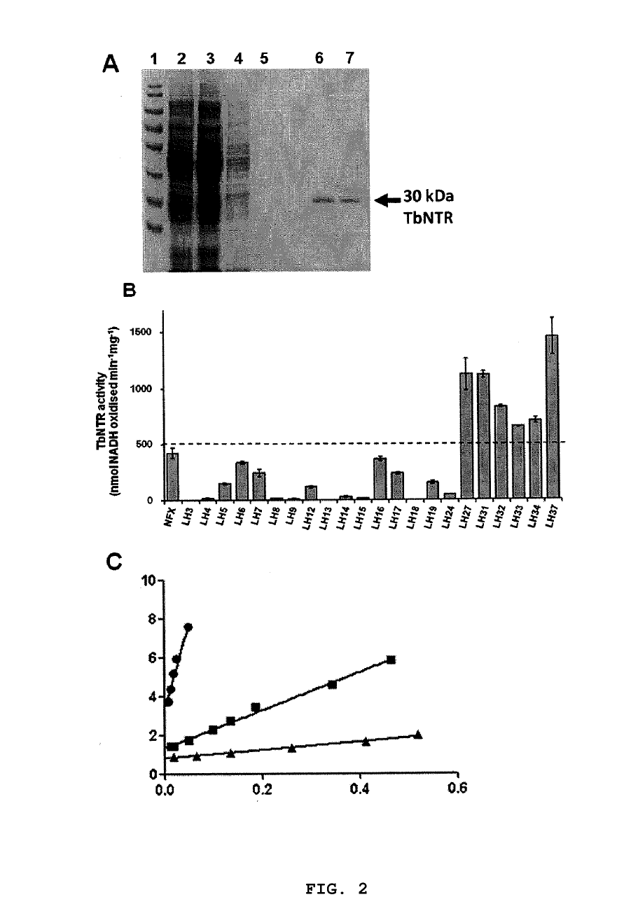

FIG. 2 depicts the activity of TbNTR toward different nitrobenzyl phosphoramide mustards. A. SDS/PAGE gel (10%) stained with Coomassie Blue. Lane 1, size standards; lane 2, crude E. coli extract loaded on to a Ni-NTA column; lane 3, flow through. The column was washed extensively with 50 mM (lane 4) and 100 mM imidazole (lane 5). Recombinant protein was eluted with 500 mM imidazole containing 0.5% Triton X-100 (lanes 6 and 7). B. The activity of purified his-tagged TbNTR was assessed using various NBPMs as substrates and the values shown are the means from three experiments±standard deviation. ThNTR activity was deemed to be high if >500 nmol NADH oxidised min⁻¹ mg⁻¹. The activity obtained when using nifurtimox (NFX) as substrate is also shown. C. TbNTR activity was assayed by following oxidation of NADH (100 μM) in the presence of TbNTR (20 μg ml⁻¹) and nitroheterocyclic substrate (2-75 μM). The substrates used were nifurtimox (), LH34 (▪) or LH 37 (▴). All reactions were initiated by the addition of the recombinant enzyme. TbNTR activities are expressed as nmol NADH oxidized min⁻¹ mg⁻¹ of enzyme.

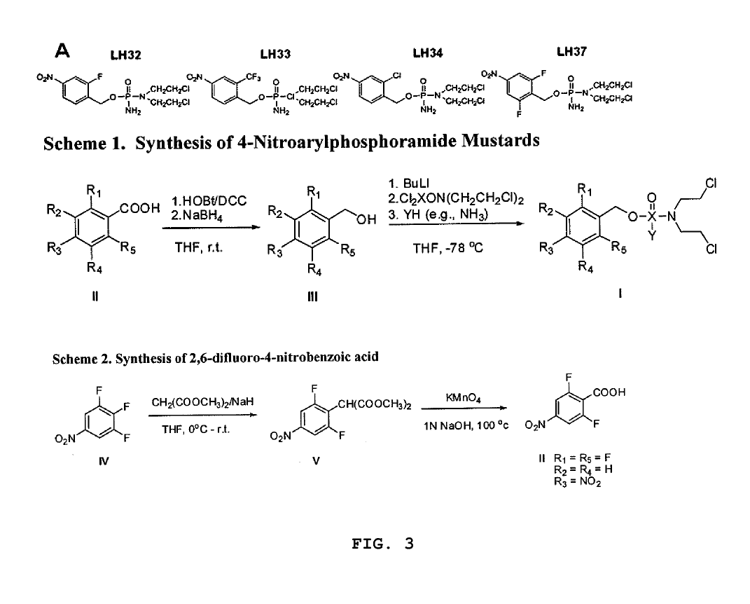

FIG. 3 depicts the susceptibility of bloodstream form TT brucei with altered levels of NTR to nitrobenzyl phosphoramide mustards. A. Structures of the NBPMs with highest trypanocidal activity (Table 3). B. Growth inhibitory effect of LH32, 33, 34 and 37 on T. brucei NTR heterozygote (NTR^{-/+}) cells as judged by IC₅₀. B. Over expression of TbNTR (TbNTR^{RV}) confers hypersensitivity to NBPMs. Data in panels A and B are means from 4 experiments±SD and the differences in susceptibility were statistically significant (p<0.01), as assessed by Student's t-test. Melarsoprol and nifurtimox were used as drug controls.

Nitroheterocyclic prodrugs have been used to treat trypanosomal diseases for more than forty years. These molecules include a nitro group linked to an aromatic ring (11) and encompass a range of molecules.

Nitroheterocyclic compounds include, e.g., the broad-spectrum nitrofuran and nitroimidazole antibiotics which are effective against a variety of urinary or digestive tract infections.

In Europe and USA, the use of nitrofuran-based compounds in food producing animals has been discontinued and their use against human infections is limited (13, 31). However, in light of emerging resistance to current therapies there is a case for reinstating nitrofurans as a front-line treatment for urinary tract infections (2, 18). Elsewhere in the World, these drugs are commonly prescribed.

Nifurtimox and benznidazole, are two nitroheterocyclic drugs, which are used to treat Chagas disease. They are orally administered and are readily absorbed from the gastrointestinal tract. Additionally, nifurtimox can cross the blood-brain barrier and recently a nifurtimoxeflornithine combination therapy (NECT) for HAT has been added to WHO Essential Medicines List (5, 25) (www.dndi.org). This, in conjunction with reports that several new nitroheterocycles have trypanocidal activity, has re-stimulated interest in this previously neglected class of compounds (3, 28).

Recently, there has been a renaissance in other nitroheterocycles. Several are currently undergoing evaluation for treatments of infectious organisms, including the nitric oxide-generating prodrug PA-824 targets Mycobacterium tuberculosis and nitazoxanide against Giardia and Cryptosporidium (1.29). Others, such as the dinitroaziridinylbenzamides, dinitrobenzamide mustards and nitrobenzylcarbamates have shown promise as anticancer therapies (7, 12).

The nitroaromatic compounds function as prodrugs and must undergo activation before mediating their cytotoxic effects. The key step in this process involves reactions catalysed by a group of oxidoreductases called nitroreductases (NTR).

Based on oxygen-sensitivity, NTRs can be divided into two groups (24,26)-Type I NTRs and Type II NTRs. Type I NTRs are oxygen-insensitive and contain FMN as co-factor. They are associated with bacteria and are absent from most eukaryotes, with a subset of protozoan parasites being major exceptions (23, 34). This difference in NTR distribution between the pathogens and human host forms the basis for the drug-selectivity of nitroheterocyclic prodrugs. Type I NTRs mediate the sequential reduction of the nitro group via a series of 2-electron transfers from NAD(P)H through a nitroso intermediate to produce hydroxylamine derivatives. It has been proposed that the hydroxylamine can generate nitrenium cations that promote DNA breakage (21, 30).

Type II NTRs are ubiquitous oxygen-sensitive enzymes that contain FAD or FMN as cofactor. They function by mediating 1-electron reduction of the nitro group that forms an unstable nitro-radical. In the presence of oxygen, this radical undergoes futile cycling to produce superoxide, with subsequent regeneration of the parent nitro-compound (8, 22).

Nitroaromatic drugs can undergo both activation events, but bacteria resistant to such agents invariably acquire mutations in their type I NTR complement, indicating that these enzymes mediate the major antimicrobial activation (20, 33).

During the type I NTR mediated conversion of the nitro group to the hydroxylamine derivative, a redistribution of electrons occurs within the nitroaromatic backbone (6).

Nitrobenzyl phosphoramide mustards are a new class of phosphoramide mustard analogues currently under investigation as anticancer agents (15-17). In these molecules, a nitroaromatic group has been incorporated as the trigger for reductive activation. Reduction of the nitro group to the hydroxylamine derivative, in a reaction catalysed by a type I NTR, leads to fragmentation of the structure, releasing toxic moieties (6). However, most eukarotic organisms lack type I NTR activity. Trypanosomes are one of the few eukaryotes to express a type I NTR.

It has been shown that the key step involved in the activation of nitrobenzyl phosphoramide mustards (NBPM) is catalyzed by a type I NTR. This class of enzyme is normally associated with bacteria and is absent from most eukaryotes, with trypanosomes being a major exception.

Nitrobenzyl phosphoramide mustards comprise a nitrobenzyl group linked to a phosphoramide mustard moiety that is derived from the anticancer drug cyclophosphamide. The linkage between these two components is key to their activity. After reduction by NTR, the NBPM hydroxylamine derivative donates electrons to the benzene ring causing an electronic rearrangement. This promotes cleavage of the benzylic C—O bond found in the para position with respect to the nitro group to produce two potent alkylating centres (FIG. 1): an aza quinine methide and the phosphoramide mustard. Initial studies have shown that the NBPM have promise as anticancer agents in gene directed prodrug therapies (15, 16, 19, 27).

There are several classes of NBPMs, differing in the nature of the linkage between the nitrobenzyl group and the phosphoramide mustard (15-17). In one set of compounds, the phosphoramide is part of a cyclic structure analogous to cyclophosphamide. Examples of these compounds are listed in Table 1.

TABLE 1
Structure of cyclic nitrobenzyl phosphoramide mustards.

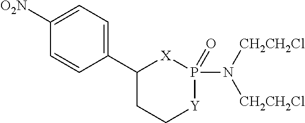

| compound | structure | diastereomer* |
|---|---|---|
| LH3 | X = O; Y = NH | cis |
| LH4 | X = O; Y = NH | trans |
| LH5 | X = NH; Y = O | cis |
| LH6 | X = NH; Y = O | trans |
| LH12 | X = NH; Y = NH | cis |
| LH13 | X = NH; Y = NH | trans |

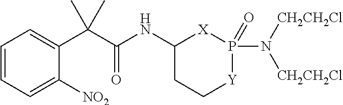

| compound | structure | diastereomer* |
|---|---|---|
| LH8 | X = O; Y = NH | cis |
| LH9 | X = O; Y = NH | trans |

In the second group, the phosphoramide chain has been linearised (Table 2). These compounds are referred to as acyclic nitrobenzyl phosphoramide mustards.

Inventors identified a subset of the acyclic nitrobenzyl phosphoramide mustards, halogenated nitrobenzyl phosphoramide mustards, that are effective substrates for NTR of T. brucei (TbNTR). T. brucei NTR plays a key role in parasite killing: heterozygous lines displayed resistance to the compounds while parasites over-expressing the enzyme showed hypersensitivity.

The halogenated nitrobenzyl phosphoramide mustards of the present invention represent a novel class of anti-trypanosomal agents. Their efficacy validates the strategy of specifically targeting NTR activity to develop new therapeutics.

In certain embodiments, a halogenated nitrobenzylphosphoramide mustard according to the present invention is a compound of formula (I):

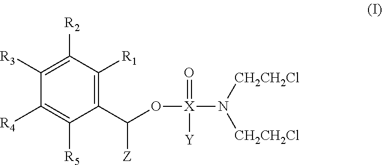

wherein:
R₁ is selected from the group consisting of hydrogen, a halogen, an amino, and a halogenated C₁-C₄ alkyl;
R₂ is selected from the group consisting of hydrogen, a halogen, an amino, and a halogenated C₁-C₄ alkyl;

### R₃ is NO₂;

R₄ is selected from the group consisting of hydrogen, a halogen, an amino, NO₂, a C₁-C₄ alkyl, a C₁-C₄ methoxyalkyl;
R₅ is selected from the group consisting of hydrogen, a halogen, an amino, NO₂, a C₁-C₄ methoxyalkyl;
Z is selected from the group consisting of hydrogen, a C₁-C₄ alkyl, a C₁-C₄ methoxyalkyl;
X is selected from the group consisting of P, C or S; and
Y is NH₂ or an aminoalkyl having 1-4 carbons.

In certain embodiments, R₁ is selected from the group consisting of hydrogen, C₁, F, Br, and CF₃; R₂ is hydrogen; R₃ is NO₂; R₄ is hydrogen; R₅ is hydrogen or Cl; Z is hydrogen; X is P or C; and Y is NH₂.

In certain embodiments, R₂ is not NO₂.

In certain embodiments, R₅ is —OCH₃.

In certain embodiments, a compound of formula (I) has a halogen in the R₁ position. The halogen may be Cl, Br or F.

In certain embodiments, a compound of formula (I) has a halogen in the R₁ and R₅ positions.

In certain embodiments, a nitrobenzyl phosphoramide mustard according to the present invention is a compound of formula (II):

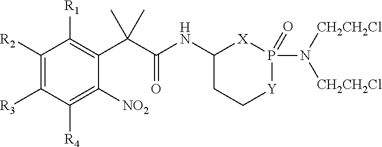

wherein:

X is O; and

Y is NH

R₁, R₂, R₃ and R₄ are each independently selected from the group consisting of hydrogen, a halogen and a halogenated C₁-C₄ alkyl.

A non-limiting list of compounds of formula (I) is provided in Table 2.

TABLE 2
Structure of acyclic nitrobenzyl phosphoramide mustards.

| | |
|---|---|
| | (I) |
| 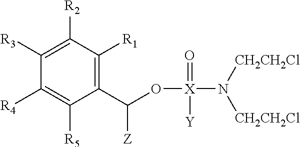 | |

| Compound | structure |
|---|---|
| LH7 | R₃ = NO₂; X = P; Y = NH₂; R₁ = R₂ = R₄ = R₅ = Z = H |
| LH14 | R₃ = NO₂; X = P; Y = NH₂; Z = CH₃; R₁ = R₂ = R₄ = R₅ = H |
| LH15 | R₅ = NO₂; X = P; Y = NH₂; R₁ = R₂ = R₄ = R₅ = Z = H |
| LH16 | R₄ = NO₂; X = P; Y = NH₂; R₁ = R₂ = R₃ = R₅ = Z = H |
| LH17 | R₃ = NO₂; R₅ = OCH₃; X = P; Y = NH₂; R₁ = R₂ = R₅ = Z = H |
| LH18 | R₃ = NO₂; R₄ = OCH₃; X = P; Y = NH₂; R₁ = R₂ = R₅ = Z = H |
| LH19 | R₃ = NO₂; R₄ = CH₃; X = P; Y = NH₂; R₁ = R₂ = R₅ = Z = H |
| LH24 | R₃ = NO₂; X = P; Y = NH₂; Z = CH₃; R₁ = R₂ = R₄ = R₅ = H |
| LH27 | R₃ = NO₂;; X—Y = C; R₁ = R₂ = R₄ = R₅ = Z = H |
| LH31 | R₂ = F; R₃ = NO₂; X = P; Y = NH₂; R₁ = R₄ = R₅ = Z = H |
| LH32 | R₁ = F; R₃ = NO₂; X = P; Y = NH₂; R₂ = R₄ = R₅ = Z = H |
| LH33 | R₁ = CF₃; R₃ = NO₂; X = P; Y = NH₂; R₂ = R₄ = R₅ = Z = H |
| LH34 | R₁ = Cl; R₃ = NO₂; X = P; Y = NH₂; R₂ = R₄ = R₅ = Z = H |
| LH37 | R₁ = R₅ = F; X = P; R₃ = NO₂; Y = NH₂; R₂ = R₄ = Z = H |
| LH47 | R₁ = R₂ = R₄ = R₅ = F; X = P; R₃ = NO₂; Y = NH₂; Z = H |
| LH48 | R₁ = R₄ = R₅ = F; X = P; R₃ = NO₂; R₂ = Y = NH₂; Z = H |

In certain embodiments, Y and Z form a ring structure and consist of —CH₂—CH₂—NH—.

In certain embodiments, the trypanocidal agent is LH7.

In certain embodiments, the trypanocidal agent is LH17.

In certain embodiments, the trypanocidal agent is LH31.

In certain embodiments, the trypanocidal agent is LH32.

In certain embodiments, the trypanocidal agent is LH33.

In certain embodiments, the trypanocidal agent is LH34.

In certain embodiments, the trypanocidal agent is LH37.

In certain embodiments, the trypanocidal agent is LH47.

In certain embodiments, the trypanocidal agent is LH48.

In certain embodiments, compound of formula I has trypanocidal activity against Trypanosoma brucei gambiense, Trypanosoma brucei rhodesiense Trypanosoma brucei brucei, Trypanosoma congolense, Trypanosoma vivax, Trypanosoma cruzi, Leishmania species, Giardia, Entamoeba, Trichomonas, and/or an additional organism expressing Type I NTR.

The compounds of the general formula I can be synthesized by using, but not limited to, a general strategy as shown in Scheme 1 through the reduction of the corresponding activated esters of suitably substituted benzoic acids (II) and subsequent phosphorylation/acylation/sulfonylation of the resulting substituted benzyl alcohol III in the presence of a base (and final amidation with ammonia in the case of phosphoramidates).

For some of the substituted benzoic acids II that are not commercially available, they can be synthesized from malonate substitution of halogenated benzenes IV followed decarboxylation and oxidation of the substituted phenyl malonate intermediate V as shown in Scheme 2.

When tested against T. brucei, cytotoxicity of these newly identified compounds mirrored enzyme activity with IC₅₀ values of the most potent substrates being less than 10 nM. The relative toxicity of these newly identified compounds was much lower than nifurtimox, based on the evaluation of the cytotoxicity of substrates using mammalian THP-1 cells.

These halogenated nitroaromatic compounds have apparent K_{cat}/Kₘ values approximately 100 times greater than nifurtimox.

The compounds covered in the present invention could also be effective against other eukaryotic parasites that express type I NTR including, but not limited to, following Trypanosoma, Leishmania, Giardia, Entamoeba, and Trichomonas: Trypanosoma brucei gambiense and Trypanosoma brucei rhodesiense—the causative agents of human infective West and East African trypanosomasis respectively.

Trypanosoma brucei brucei—causes Nagana in cattle & is used as a model organism for the human African trypanosomasis (this is the organism we have used).

Trypanosoma congolense—causes Nagana in cattle and other animals including sheep, pigs, goats, horses and camels.

Trypanosoma vivax—causes Nagana in cattle.

Trypanosoma cruzi—the causative agents of human infective Chagas disease.

Leishmania species—there are numerous different species (21 species infect humans) that cause a spectrum of infections including cutaneous leishmnaiasis, visceral leishmnaiasis and mucocutaneous leishmnaiasis.

Giardia, Entamoeba, and Trichomonas which cause diarrhea, dysentery, and vaginitis, respectively.

The compounds of the present invention can be administered alone or can be combined with various pharmaceutically acceptable carriers and excipients known to those skilled in the art, including but not limited to diluents, suspending agents, solubilizers, binders, disintegrants, preservatives, coloring agents, lubricants and the like.

The compounds of the present invention may also be incorporated into liquid dosage forms, including aqueous and nonaqueous solutions, emulsions, suspensions, and solutions and/or suspensions reconstituted from non-effervescent granules, containing suitable solvents, preservatives, emulsifying agents, suspending agents, diluents, sweeteners, coloring agents, and flavoring agents.

The compounds of the present invention may be administered as a parenteral dosage form. When the compounds are incorporated for parenteral administration by injection (e.g., continuous infusion or bolus injection), the formulation for parenteral administration may be in the form of suspensions, solutions, emulsions in oily or aqueous vehicles, and such formulations may further comprise pharmaceutically necessary additives such as, e.g., stabilizing agents, suspending agents, dispersing agents, and the like.

The compounds of the invention may also be in the form of a powder for reconstitution-as an injectable formulation.

When the compounds of the present invention are to be injected parenterally, they may be, e.g., in the form of an isotonic sterile solution.

The compounds of the present invention may be incorporated into various oral dosage form, including such solid forms as tablets, gelcaps, capsules, caplets, granules, lozenges and bulk powders and liquid forms such as, e.g., emulsions, solution and suspensions.

When the compounds of the present invention are incorporated into oral tablets, such tablets can be compressed, tablet triturates, enteric-coated, sugar-coated, film-coated, multiply compressed or multiply layered.

In addition, when the compounds of the present invention are incorporated into oral dosage forms, it is contemplated that such dosage forms may provide an immediate release of the compound in the gastrointestinal tract, or alternatively may provide a controlled and/or sustained release through the gastrointestinal tract. A wide variety of controlled and/or sustained release formulations are well known to those skilled in the art, and are contemplated for use in connection with the formulations of the present invention. The controlled and/or sustained release may be provided by, e.g., a coating on the oral dosage form or by incorporating the compound(s) of the invention into a controlled and/or sustained release matrix.

The compounds of the present invention are to be inhaled, they may be formulated into a dry aerosol or may be formulated into an aqueous or partially aqueous solution.

Specific examples of pharmaceutically acceptable carriers and excipients that may be used to formulate dosage forms of the present invention, are described in the Handbook of Pharmaceutical Excipients, American Pharmaceutical Association (1986).

Techniques and compositions for making solid oral dosage forms are described in Pharmaceutical Dosage Forms: Tablets (Lieberman, Lachman and Schwartz, editors) 2nd edition, published by Marcel Dekker, Inc. Techniques and compositions for making tablets (compressed and molded), capsules (hard and soft gelatin) and pills are also described in Remington's Pharmaceutical Sciences (Arthur Osol, editor), 1553B1593 (1980). Techniques and composition for making liquid oral dosage forms are described in Pharmaceutical Dosage Forms: Disperse Systems, (Lieberman, Rieger and Banker, editors) published by Marcel Dekker, Inc.

The compounds and compositions of the invention can be enclosed in multiple or single dose containers. The enclosed compounds and compositions can be provided in kits, for example, including component parts that can be assembled for use. The kit can also optionally include instructions for use in any medium. For example, the instructions can be in paper or electronic form. For example, a compound of the present invention in lyophilized form and a suitable diluent may be provided as separated components for combination prior to use. A kit may include a compound of the present invention and a second therapeutic agent for co-administration. The compound of the present invention and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The compounds of the invention can be used in combination, with each other or with other therapeutic agents or approaches used to treat protozoan infections.

### DETAILED DESCRIPTION OF THE PREFERRED EMBODIMENTS

### Example 1

### Preparation of 2,6-difluoro-4-nitrobenzylmalonic acid dimethyl ester (V)

At 0° C., to a solution of dimethyl malonate (2.3 mL, 20.0 mmol) in anhydrous THF (20 mL) was added 60% sodium hydride (0.8 g, 20.0 mmol). The resulting mixture was stirred at 0-5° C. for 1 h followed by addition of 3,4,5-trifluoro-nitrobenzene (1.77 g, 10.0 mmol). The reaction was then carried out at room temperature for 24 h and was quenched by addition of 5 ml 10% HCl. After evaporation of THF under reduced pressure, the residue was redissolved in 100 ml of EtOAc. The organic phase was then washed with water and brine, and dried over Na₂SO₄. After removal of Na₂SO₄ via filtration, the filtrate was concentrated to dryness. The crude product was cyrstallized from hexane, affording the desired product 2,6-difluoro-4-nitrobenzylmalonic acid dimethyl ester (2.1 g) as a white solid in a yield of 71%. ¹H NMR (200 MHz, CDCl₃): δ7.80 (d, 2H, J=7.0 Hz), 5.00 (s, 1H), 3.77 (s, 3H); ¹³C NMR (50 MHz, CDCl₃): 165.7, 160.7 (dd, J=255.0 Hz, J=8.0 Hz), 148.5 (t, J=11.0 Hz), 117.5 (t, J=19.0 Hz), 107.8 (dd, J=28.0 Hz, J=3.0 Hz), 53.4, 41.0; MS (ESL): m/z (intensity), 288.0 ([M−H]⁻, 100%).

### Synthesis of 2,6-difluoro-4-nitrobenzoic acid (II)

At 50° C., to a solution of V (2.0 g, 6.92 mmol) in 0.5 N NaOH (56 mL) was added potassium permanganate (5.47 g, 34.60 mmol) portionwise over 1 h. After addition of all potassium permanganate, the resulting reaction mixture was stirred at reflux for additional 2 h. The mixture was then passed through a Celite pad while still hot. The brown Celite pad was rinsed with hot water (2×50 mL). The combined aqueous phase was acidified by conc. HCl to pH-1 followed by extraction with EtOAc (3×50 mL). The combined organic phase was then washed with water and brine, and dried over Na₂SO₄. After removal of Na₂SO₄ via filtration, the filtrate was concentrated to dryness. The crude product was purified by flash chromatography (hexane to EtOAc) to afford the desired product 2,6-difluoro-4-nitrobenzoic acid (1.1 g) as a white solid in a yield of 78%. 'H NMR (200 MHz, Acetone-d₆): δ 9.24 (brs, —COOH), 8.04 (d, 2H, J=7.4 Hz); ¹³C NMR (50 MHz, Acetone-d₆): 160.6, 160.3 (dd, J=255.0 Hz, J=8.0 Hz), 150.4 (t, J=12.0 Hz), 118.0 (t, J=20.0 Hz), 108.9 (dd, J=28.0 Hz, J=4.0 Hz); MS (ESI′): m/z (intensity), 405.1 ([2M−H]⁻, 100%), 158.0 ([M-COON]⁻, 50%).

### General Method for the Synthesis of 4-Nitro-Benzyl Alcohols (III)

To a solution of substituted 4-nitrobenzoic acid II (1.0 mmol) in THF (10 mL) was added HOBt (135 mg, 1.0 mmol) and DCC (227 mg, 1.1 mmol). The resulting mixture was stirred at room temperature for 1 h. The white precipitates were filtered off and rinsed with THF (2×5 mL). The filtrate was added dropwise to a suspension of NaBH₄ (38 mg, 1.0 mmol) in THF (10 mL) over 30 min. The reaction was carried out at room temperature for additional 1.5 h and quenched by adding 10 mL 1.0 N HCl. After evaporation of THF under reduced pressure, the remaining aqueous solution was extracted EtOAc (3×20 mL). The combined EtOAc phase was washed with water and brine, and dried over Na₂SO₄. After removal of Na₂SO₄ via filtration, the filtrate was concentrated to dryness. The crude product was purified by flash column chromatography (hexane to 50% EtOAc/hexane) to afford the desired alcohol III.

### 3-Fluoro-4-nitrobenzyl alcohol

A yellow solid (161 mg, 94%); ¹H NMR (200 MHz, CDCl₃): δ 8.01 (t, 1H, J=7.8 Hz), 7.29 (d, 1H, J=11.8 Hz), 7.22 (d, 1H, J=8.4 Hz), 4.77 (s, 2H), 2.00 (s, —OH); ¹³C NMR (50 MHz, CDCl₃): 155.7 (d, J=265.0 Hz), 150.3 (t, J=8.0 Hz), 136.9 (t, J=8.0 Hz), 126.2 (d, J=2.0 Hz), 121.7 (d, J=4.0 Hz), 115.8 (d, J=21.0 Hz), 63.4; 1R (KBr): 2146, 1691, 1432, 1298 cm⁻¹.

### 2-Fluoro-4-nitrobenzyl alcohol

A yellow solid (161 mg, 94%); ¹H NMR (200 MHz, CDCl₃): δ8.06 (dd, 1H, J=8.4 Hz, J=1.8 Hz), 7.90 (dd, 1H, J=9.6 Hz, J=1.8 Hz), 7.70 (t, 1H, J=7.8 Hz), 4.78 (s, 2H), 2.16 (s, —OH); ¹³C NMR (50 MHz, CDCl₃): 159.3 (d, J=250.0 Hz), 148.1 (d, J=5.0 Hz), 135.5 (d, J=15.0 Hz), 129.0 (d, J=5.0 Hz), 119.4 (d, J=4.0 Hz), 110.9 (d, J=27.0 Hz), 58.4 (d, J=4.0 Hz); IR (KBr): 3524, 1516, 1355, 1051 cm⁻¹.

### 2,6-Difluoro-4-nitrobenzyl alcohol

A yellow solid (164 mg, 87%); ¹H NMR (200 MHz, CDCl₃): δ 8.02 (d, 2H, J=6.8 Hz), 5.05 (s), 2.31 (s, —OH); ¹³C NMR (50 MHz, CDCl₃): 161.7 (dd, J=252.0 Hz, J=9.0 Hz), 148.0 (t, J=10.0 Hz), 123.7 (t, J=20.0 Hz), 108.4 (dd, J=30.0 Hz, J=2.0 Hz), 53.4 (t, J=4.0 Hz); IR (KBr): 2146, 1691, 1432, 1298 cm⁻¹.

### 2-Chloro-4-nitrobenzyl alcohol

A yellow solid (172 mg, 92%); ¹H NMR (200 MHz, CDCl₃): 8.14 (d, 1H, J=2.2 Hz), 8.08 (dd, 1H, J=8.0 Hz, J=2.2 Hz), 7.72 (d, 1H, J=8.0 Hz), 4.81 (s), 2.78 (s, —OH); ¹³C NMR (50 MHz, CDCl₃): 147.9, 146.4, 133.1, 128.7, 124.8, 122.5, 62.4; IR (KBr): 2146, 1691, 1432, 1298 cm⁻¹.

### 2-Trifluoromethyl-4-nitrobenzyl alcohol

A yellow solid (201 mg, 91%); ¹H NMR (200 MHz, CDCl₃): δ8.44 (s, 1H), 8.36 (d, 1H, J=8.4 Hz), 8.01 (d, 1H, J=8.4 Hz), 4.97 (s), 2.69 (s, —OH); ¹³C NMR (50 MHz, CDCl₃): 146.9, 146.5, 129.1, 128.0 (d, J=32.0 Hz), 126.7, 122.9 (q, J=270.0 Hz), 121.2 (q, J=6.0 Hz), 60.3 (d, J=3.0 Hz); IR (KBr): 2146, 1691, 1432, 1432, 1298 cm⁻¹

### General Method for the Synthesis of 4-Nitro-Arylmethyl Phosphoramide Mustards

To a solution of the alcohol III (1.0 mmol) in anhydrous THF (5 mL) was added a solution of BuLi in cyclohexane (2.0 M, 0.55 mL) at −78° C. After 20 min, the above solution was transferred to a pre-cooled solution of bis(2-chloroethyl)phosphoramidic dichloride (285 mg, 1.1 mmol) in THF (5 mL) at −78° C. via a cannula. The resulting mixture was stirred at −78° C. for 5 h followed by bubbling with ammonia for 10 min. The reaction mixture was allowed to gradually warm up to room temperature during the period of 2 h. After removal of THF via distillation under reduced pressure, the residue was suspended in saturated aqueous sodium bicarbonate (10 mL) followed by extraction with dichloromethane (3×25 mL). The combined organic phase was washed with water (25 mL) and saturated brine (25 mL), and dried over Na₂SO₄. After filtration off Na₂SO₄, the filtrate was concentrated to dryness under reduced pressure. The crude product was purified by flash column chromatography (dichloromethane to 5% methanol in dichloromethane) to afford the desired product I.

### 3-Fluoro-4-nitrobenzyl phosphoramide mustard (LH31)

A yellow solid (206 mg, 55%); ¹H NMR (200 MHz, CDCl₃): δ 8.00 (t, 1H, J=7.8 Hz), 7.27 (d, 1H, J=12.4 Hz), 7.22 (d, 1H, J=8.0 Hz), 5.03 (t, 2H, J=7.2 Hz), 3.56-3.63 (m, 4H), 3.40-3.49 (m, 4H), 2.97 (s, —NH₂); ¹³C NMR (50 MHz, CDCl₃): 155.6 (d, J=265.0 Hz), 145.7 (d, J=8.0 Hz), 136.9 (t, J=8.0 Hz), 126.4 (d, J=2.0 Hz), 122.6 (d, J=4.0 Hz), 116.6 (d, J=22.0 Hz), 65.0, 48.8 (d, J=5.0 Hz), 42.5; IR (KBr): 2146, 1691, 1432, 1298 cm⁻¹. HRMS (FAB⁺) m/z calc'd for C₁₁H₁₅Cl₂FN₃O₄P, [M+H]⁺, as 374.0240, found 374.0229.

### 2-Fluoro-4-nitrobenzyl phosphoramide mustard (LH32)

A yellow solid (183 mg, 49%); ¹H NMR (200 MHz, CDCl₃): δ 7.96 (d, 1H, J=8.4 Hz), 7.84 (d, 1H, J=9.6 Hz), 7.63 (t, 1H, J=7.8 Hz), 5.08 (d, 2H, J=7.0 Hz), 3.46-3.61 (m, 4H), 3.34-3.46 (m, 4H); ¹³C NMR (50 MHz, CDCl₃): 159.3 (d, J=250.0 Hz), 148.2 (d, J=9.0 Hz), 131.4 (dd, J=14.0 Hz, J=8.0 Hz), 129.8 (d, J=5.0 Hz), 119.3 (d, J=4.0 Hz), 111.0 (d, J=26.0 Hz), 60.1 (t, J=4.0 Hz), 48.8 (d, J=5.0 Hz), 42.3; IR (KBr): 2146, 1691, 1432, 1298 cm⁻¹. HRMS (FAB⁺) m/z calc'd for C₁₁H₁₅Cl₂FN₃O₄P, [M+H]⁺, as 374.0240, found 374.0235.

### 2,6-Difluoro-4-nitrobenzyl phosphoramide mustard (LH37)

A yellow solid (157 mg, 40%); ¹H NMR (200 MHz, CDCl₃): δ 7.81 (d, 2H, J=6.6 Hz), 5.13 (t, J=6.8 Hz), 3.59-3.66 (m, 4H), 3.38-3.50 (m, 4H), 2.92 (s, —NH₂); ¹³C NMR (50 MHz, CDCl₃): 160.4 (dd, J=255.0 Hz, J=8.0 Hz), 148.2 (t, J=10.0 Hz), 118.7 (td, J=18.0 Hz, J=8.0 Hz), 107.0 (dd, J=30.0 Hz, J=2.0 Hz), 53.6 (d, J=4.0 Hz), 48.2 (d, J=5.0 Hz), 41.6; IR (KBr): 2146, 1691, 1432, 1298 cm⁻¹; MS (EST⁺): m/z (intensity), 392.0 ([M+H]⁺, 100%), 394.0 ([M+H]⁺+2.65%), 396.0 ([M+H]⁺+4.10%). HRMS (FAB⁺) m/z calc'd for C₁₁, H₁₄Cl₂F₂N₃O₄P, [M+H]⁺, as 392.0145, found 392.0142.

### 2-Chloro-4-nitrobenzyl phosphoramide mustard (LH34)

A yellow solid (250 mg, 64%). ¹H NMR (200 MHz, CDCl₃): δ 8.14 (d, 1H, J=2.2 Hz), 8.08 (dd, 1H, J=8.0 Hz, J=2.2 Hz), 7.72 (d, 1H, J=8.0 Hz), 5.12 (d, J=7.0 Hz), 3.58-3.68 (m, 4H), 3.42-3.50 (m, 4H), 3.36 (brs, —NH₂); ¹³C NMR (50 MHz, CDCl₃): 147.6, 141.7 (d, J=9.0 Hz), 132.9, 128.6, 124.3, 121.8, 63.4 (d, J=3.0 Hz), 48.8 (d, J=5.0 Hz), 42.3; IR (KBr): 2146, 1691, 1432, 1298 cm⁻¹. HRMS (FAB⁺) m/z calc'd for C₁₁H₃Cl₃N₃O₄P, [M+H]⁺, as 389.9944, found 389.9943.

### 2-Trifluoromethyl-4-nitrobenzyl phosphoramide mustard (LH33)

A yellow solid (220 mg, 52%); ¹H NMR (200 MHz, CDCl₃): δ 8.49 (s, 1H), 8.40 (d, 1H, J=8.4 Hz), 7.93 (d, 1 H, J=8.4 Hz), 5.27 (d, 1H, J=6.6 Hz), 3.60-3.66 (m, 4H), 3.42-3.53 (m, 4H), 3.21 (brs, —NH₂); ¹³C NMR (50 MHz, CDCl₃): 147.1, 142.3 (d, J=10.0 Hz), 129.9, 128.6 (d, J=32.0 Hz), 126.8, 122.7 (q, J=273.0 Hz), 121.5 (q, J=6.0 Hz), 60.3 (t, J=3.0 Hz), 48.7 (d, J=5.0 Hz), 42.3; IR (KBr): 2146, 1691, 1432, 1298 cm⁻¹. HRMS (FAB⁺) m/z calc'd for C₁₂H₁₅Cl₂F₃N₃O₄P, [M+H]⁺, as 424.0208, found 424.0172.

### Example 2

A comparison of 22 NBPMs was performed to determine whether there is a relationship between trypanosomal type I NTR activity and in vitro activity against bloodstream form parasites. Two of these compounds were highly active against T. brucei and displayed high selectivity toward the parasite. Compared against the existing nifurtimox and benznidazole therapies, NBPMs appear to be a promising new class of trypanocidal agents.

Materials and Methods

Chemicals

Structures of nitrobenzyl phosphoramide mustards synthesized are shown in Tables 1 and 2 (15-17). All compounds were fully characterized using NMR and MS and their purity judged to be >90%: most were >95% based on LC-MS analysis.

Cell Culturing

T. brucei (MITat 427 strain; clone 221 a) bloodstream (BSF) trypomastigotes were grown at 37° C. under a 5% CO₂ atmosphere in modified Iscove's medium as previously described (14). Transformed parasite lines containing altered levels of TbNTR were maintained in this medium supplemented with either 2 μg ml⁻¹ puromycin (for heterozygous NTR⁻⁴ cells) or 2.5 μg ml⁻¹ hygromycin/1 μg ml⁻¹ phleomycin (for ThNTR over expressing cells) (34). Tetracycline free foetal calf serum (Autogen Bioclear) was used in the growth media.

A human acute monocytic leukemia cell line (THP-1) was grown at 37′C under a 5% CO₂ atmosphere in RPMI 1640 medium supplemented with 10% tetracycline free foetal calf serum, 20 mM HEPES pH 8.0, 2 mM sodium glutamate, 2 mM sodium pyruvate, 2.5 U ml⁻¹ penicillin, 2.5 μg ml⁻¹, streptomycin.

Anti-Proliferative Assays.

T. brucei BSF parasites were seeded at 1×10³ ml⁻¹ in 200 μl growth medium containing different concentrations of NBPM. Where appropriate, induction was carried out by adding tetracycline (1 μg ml⁻¹). After incubation at 37° C. for 3 days, 20 μl Alamar blue (Biosource UK Ltd) was added to each well and the plates incubated for a further 16 hours. The fluorescence of each culture was determined using a Gemini Fluorescent Plate reader (Molecular Devices) at an excitation wavelength of 530 run, emission wavelength of 585 rim and a filter cut off at 550 run. The colour change resulting from the reduction of Alamar blue is proportional to the number of live cells. The IC₅₀ value for each compound was then established.

Growth inhibition of T. cruzi amastigotes was monitored as follows. Vero cells were seeded at 1.5×10⁴ ml⁻¹ in 100 μl in growth medium and allowed to adhere to the well for 6 hrs. T. cruzi trypomastigotes (10,000 in 100 μl growth medium) were then added to each well and infections performed overnight at 37° C. under a 5% CO₂. The cultures were then washed twice in growth medium to remove non-internalised parasites and the supernatant replaced with fresh growth medium containing drug. Drug-treated infections were incubated for a further 3 days at 37° C. under a 5% CO₂. The growth medium was then removed and the cells lysed in 50 μl cell culture lysis reagent (Promega). Activity was then measured using the luciferase assay system (Promega) and light emission measured on a 13-plate counter (Wallac). The luminescence is proportional to the number of live cells. The IC₅₀ value for each compound was then established.

Growth inhibition of L. major promastigotes was monitored as follows. Parasites were seeded at 5×10⁵ ml⁻¹ in 200 μl growth medium containing different concentrations of drug. After incubation at 25° C. for 6 days, 20 μl Alamar blue (Biosource UK Ltd) was added to each well and the plates incubated for a further 16 hours. The fluorescence of each culture was determined using a Gemini Fluorescent Plate reader (Molecular Devices) at an excitation wavelength of 530 nm, emission wavelength of 585 nm and a filter cut off at 550 nm. The colour change resulting from the reduction of Alamar blue is proportional to the number of live cells. The IC₅₀ value for each compound was then established.

To assess mammalian cell cytotoxicity, THP-1 or Vero cells were seeded at 1×10⁴ ml⁻¹ in 200 μl growth medium containing different concentrations of compound. After incubation at 37° C. for 6 days, 20 μl Alamar blue (Biosource UK Ltd) was added to each well and the plates incubated for a further 8 hours. The cell density of each culture was determined as described above and the IC₅₀ value established.

Protein Purification and Enzyme Assay.

A DNA fragment encoding for the catalytic domain of T. brucei NTR (TbNTR) was amplified from genomic DNA with the primers ggatccTTGATGCATTTATACGTGTTG and gaattcTCAGAAGCGATTCCATCGGAC; lower-case italics correspond to restriction sites incorporated into the primers to facilitate cloning. The fragment was digested with BamHI/HindIII then cloned into the corresponding sites of the vector pTrcHis-C (Invitrogen). A 2 litre E. coli BL-21+ pTrcHis-TbNTR culture was grown at 37° C. for 2 hours with aeration. Protein expression was then induced by IPTG, the culture incubated overnight at 16° C. and the cells harvested by centrifugation. His-tagged TbNTR was affinity purified on a Ni-NTA column (Qiagen) and eluted with 500 mM imidazole; 500 mM NaCl; 50 mM NaHPO₄ pH 7.8. The elution steps were carried out in the presence of 0.5% Triton X-100 and protease inhibitors (Roche). Fractions were analysed by SDS-PAGE and protein concentrations determined by BCA protein assay system (Pierce).

Enzyme activity was measured by following the change in absorbance at 340 nm due to NADH oxidation (34, 35). A reaction mixture (1 ml) containing 50 mM Tris-Cl pH 7.0, 100 μM NADH and NBPM (Tables 1 and 2) was incubated at room temperature for 5 min. The background rate of NADH oxidation was determined and the reaction initiated by the addition of 20 μg TbNTR. The enzyme activity was calculated using c of 6,220 M⁻¹ cm⁻¹. The absorbance spectrum (320-600 nm) for each NBPM was determined before. At the concentration ranges used no significant signal was detected at 340 nm for any of the drugs tested.

Stability Study Of Nitrobenzyl Phosphoramide Mustards in Phosphate Buffer or Whole Human Blood

Select nitrobenzyl phosphoramide mustards (100 μM) were incubated in pH 7.4 100 mM phosphate buffer or in whole human blood at 37° C. Aliquots were withdrawn at different time intervals and mixed with acetonitrile (1-1 for phosphate buffer and 1-5 for whole blood). The samples were centrifuged and analyzed by HPLC.

Results

Metabolism of Nitrobenzyl Phosphoramide Mustards by the Trypanosomal NTR.

Activation of the nitroheterocyclic prodrugs nifurtimox and benznidazole by trypanosomes is mediated by a type I NTR (34). As an initial screening strategy, the inventors determined whether recombinant T. brucei NTR displayed activity toward the library of NBPMs described in Tables 1 and 2.

The region of the ThNTR gene encoding the catalytic domain was expressed in E. coli (Materials and Methods): attempts to express the full length protein failed to generate soluble enzyme. In the system used, recombinant enzyme is tagged at its amino terminal with a histidine-rich sequence and an epitope detectable with the anti-Xpress monoclonal antibody (Invitrogen). A band of 30 kDa was detectable in bacterial lysates by western blot. The native protein could be purified by one round of affinity chromatography on a Ni-NTA column (FIG. 2A).

The activity of TbNTR toward NBPMs was followed by monitoring the change in absorbance at 340 nm corresponding to NADH oxidation (FIG. 2B). In total, 22 compounds were screened: 8 structures where the nitrobenzyl group is attached to cyclophosphamide directly or through a carbamate linker (Table 1) and 14 where phosphoramide mustard is linked to the nitrobenzyl group with varying substituents (Table 2). The cyclophosphamide analogues were poor substrates for TbNTR, but six of the linear compounds (LH27, 31-34 & 37) were shown to be “good” NTR substrates, generating an activity >500 nmol NADH oxidized min⁻¹ mg⁻¹ (nifurtimox yielded an activity of 423±45 nmol NADH oxidised min⁻¹ mg⁻¹). Further analysis with LH27 was discontinued after it was shown to lack trypanocidal activity (see below). Of the remaining 5 structures, all contained at least one halogen linked to the nitro-substituted benzene ring.

Kinetic studies were carried out to investigate the interaction of TbNTR with the NBPM compounds. Double reciprocal plots of 1/TbNTR activity against 1/[NBPM] were linear for substrate concentrations up to 75 μM (FIG. 2C). Extrapolation of the slopes allowed apparent kinetic constants for each substrate to be calculated (Table 3). TbNTR exhibited a higher affinity and activity toward all halogenated NBPM compounds compared to nifurtimox as judged by their lower apparent Kₘ, higher apparent Vₘₐₓ and catalytic efficiency values.

Trypanocidal Activity of Nitrobenzyl Phosphoramide Mustards.

To determine whether there was a correlation between biochemical activity and parasite killing, all NBPMs were initially screened for trypanocidal activity against bloodstream form T. brucei (Materials and methods). Out of the 22 compounds, 15 had no effect on parasite growth at concentrations up to 10 μM (Table 4). These were not analysed further. For the remaining 7 compounds, growth inhibition assays were performed to determine their IC₅₀ value (Table 4). All of these displayed an appreciable trypanocidal activity (<5 μM) with 4 having an IC₅₀ lower than 500 nM. Two of these compounds (LH34 and 37) had IC₅₀ values of <10 nM, more than two orders of magnitude lower than nifurtimox. The 4 compounds generating the lowest IC₅₀ values correspond to structures previously designated as ‘good’ TbNTR substrates (Table 3).

To confirm that trypanosomal NTRs play a key role in the activation of NBPM prodrugs, the inventors used T. brucei BSF cells where the level of the enzyme had been genetically altered (34). The heterozygous and over expressing cell lines were grown in different concentrations of LH32, 33, 34 or 37 (FIG. 3A) and the IC₅₀ values determined. Cells containing a single copy of the TbNTR gene (NTR^{+/-}) were up to 6-fold more resistant to the nitroaromatic structures than controls (FIG. 3B). In contrast, parasites with elevated levels of TbNTR were shown to be 10-fold more sensitive to the mustard compounds than controls (FIG. 3C). This was shown to be NTR specific, as all parasite lines when treated with melarsoprol, a normitroaromatic drug control, displayed similar drug sensitivities (approximately 4 μM).

Cytotoxicity Against Mammalian Cells.

The 7 compounds identified as having appreciable trypanocidal activity were assayed for cytotoxicity against THP-1 cells (Table 4). The therapeutic index (TI) (IC₅₀ against the mammalian line/IC₅₀ against the parasite) for each compound was then determined. In all cases, the agents displayed selective toxicity toward the parasite. For the parent, non-halogenated compound (LH7) and 2 other mustards (LH17 and 31), a TI equivalent to that determined for nifurtimox were obtained. For the 4 halogenated compounds identified as being preferred TbNTR substrates and having highest trypanocidal activity (LH32, 33, 34 and 37), higher TIs were observed. For 2 compounds, LH34 and 37, their TIs were 1250 and 986, respectively, 35 and 27-fold higher than nifurtimox. Thus, the relative toxicity of 2 of these newly identified compounds is much lower than that of nifurtimox.

TABLE 3
Activity of TbNTR toward different nitrobenzyl phosphoramide
mustards. Enzyme activity (Vmax) was calculated using an
c value of 6.22 mM⁻¹. Kcat assumes one catalytic site per
30-kDa monomer.

| | | Activity (Vmax), | |
|---|---|---|---|
| | Michaelis-Menten | nmol NADH | Catalytic |
| | Constant (Kₘ), | oxidized | efficiency |
| Substrate | μM | min⁻¹mg⁻¹ | (K_{cat}/Kₘ), M⁻¹s⁻¹ |
| Nifurtimox | 53.1 ± 15.2 | 423.3 ± 45 | 3.4 × 10³ |
| LH7 | 80.3 ± 24.5 | 245.0 ± 34.5 | 1.3 × 10³ |
| LH32 | 6.9 ± 0.5 | 832 ± 9 | 5.2 × 10⁴ |
| LH33 | 2.4 ± 0.3 | 654 ± 14 | 1.2 × 10⁵ |
| LH34 | 8.4 ± 0.6 | 706 ± 27 | 4.3 × 10⁵ |
| LH37 | 2.8 ± 0.4 | 1238 ± 48 | 1.8 × 10⁵ |

TABLE 4
Susceptibility of bloodstream form T. brucei and mammalian
cells to nitrobenzyl phosphoramide mustards.

| | IC₅₀ | | |
|---|---|---|---|
| | T. bruccei | IC₅₀ | Therapeutic |
| Compound | (μM) | THP1 (μM) | Index |
| Nifurtimox | 1.800 ± 0.4 | 64.8 ± 1.5 | 36 |
| LH3-6; LH8; LH9, | >10 | nd | nd |
| LH12-16; LH18; | | | |
| LH19; LH24; LH27 | | | |
| LH7 | 3.400 ± 0.300 | 66.0 ± 0.8 | 19 |
| LH17 | 1.200 ± 0.100 | 33.5 ± 5.8 | 28 |
| LH31 | 3.200 ± 0.200 | 17.6 ± 1.5 | 6 |
| LH32 | 0.268 ± 0.008 | 20.3 ± 1.8 | 76 |
| LH33 | 0.149 ± 0.020 | 19.0 ± 1.9 | 128 |
| LH34 | 0.008 ± 0.001 | 10.0 ± 1.2 | 1250 |
| LH37 | 0.007 0.001 | 6.9 1.3 | 986 |

TABLE 5
Susceptibility of intracellular form T. cruzi and mammalian
cells to nitrobenzylphosphoramide mustards.

| | IC₅₀ (μM) | | |
|---|---|---|---|
| Compounds | T. cruzi | Vero cells | Relative toxicity |
| LH3-9 LH12-19; | >10 | >100 | nd |
| LH24; LH27; LH31 | | | |
| LH32 | 9.54 ± 0.27 | >100 | >10 |
| LH33 | 7.97 ± 1.13 | >100 | >13 |
| LH34 | 9.14 ± 0.54 | >100 | >11 |
| LH37 | 0.99 ± 0.01 | >100 | >100 |
| LH47 | 8.69 ± 0.76 | 6.98 ± 0.69 | <1 |
| LH48 | 8.23 ± 0.30 | 64.38 ± 0.53 | 8 |

TABLE 6
Susceptibility of insect form Leishmania major and mammalian
cells to nitrobenzylphosphoramide mustards.

| | IC₅₀ (μM) | | |
|---|---|---|---|
| Compounds | L. major | Vero cells | Relative toxicity |
| LH3-9 LH12-15; | >30 | >100 | nd |
| LH17-19; LH24; | | | |
| LH16 | 15.6 ± 1.13 | >100 | >6 |
| LH27 | 16.23 ± 0.29 | >100 | >6 |
| LH31 | 9.05 ± 0.56 | >100 | >11 |
| LH32 | 4.72 ± 0.23 | >100 | >20 |
| LH33 | 5.88 ± 0.28 | >100 | >17 |
| LH34 | 3.10 ± 0.28 | >100 | >33 |
| LH37 | 1.29 ± 0.08 | >100 | >77 |
| LH47 | 5.13 ± 0.25 | 6.98 ± 0.69 | 1 |
| LH48 | 4.72 ± 0.97 | 64.38 ± 0.53 | 14 |

### Example 3

The present inventors performed a structure activity relationship study on a library of NBPMs using both biochemical and trypanocidal screens (FIG. 2B; Table 4).

TbNTR was shown to metabolise some of the cyclic phosphoramides analogues albeit at a slow rate (FIG. 2B). However, none of these killed BSF parasites in the concentration range tested (Table 4).

The acyclic nitrobenzyl phosphoramide mustard (LH7) was shown to be reduced by TbNTR at a rate similar to that of the cyclic analogues, but in contrast, this translated into a trypanocidal activity (FIG. 2B; Table 4). Based on this, only derivatives of the acyclic NBPM were evaluated further.

A second series of acyclic compounds was then evaluated. Initially, the positional affect of the nitro group on the benzyl ring in relation to the phosphoramide mustard was examined. This demonstrated that compounds with the nitro group in the 2- or 4-arrangement functioned as TbNTR substrates but only the 4-NBPM (LH7) displayed trypanocidal activity (FIG. 2B; Table 4): no TbNTR and trypanocidal activity was detected when the nitro group was in the 3-position. All subsequent compounds contained the nitro group in the 4-position. Addition of a methyl group on the benzylic carbon or benzyl ring using the LH7 structure as template resulted in compounds with a reduced enzymatic activity and parasite killing. Interestingly, a compound (LH27) where the phosphorous on the phosphoramide chain had been replaced with a carbamate was readily metabolised by TbNTR. However, for reasons that are unclear, this derivative had no effect against BSF trypanosomes. It may be inefficiently transported into the cell or unable to access the mitochondrion, the sub-cellular location where TbNTR is found. Alternatively, nitroreduction may not result in the cleavage of this particular carbamate analogue and subsequent elimination of the nitrogen mustard. However, this result does illustrate the importance of phosphoramide mustard at this position and highlights its presence as an essential requirement in this class of compounds.

With acyclic 4-NBPM (LH7) as a lead structure, the effects of substitutions that alter the electronic characteristics of the aromatic ring and the benzylic carbon were examined. Addition of an electron-donating methoxy group in the 2-position produced a substrate (LH17) with equivalent TbNTR activity to the lead compound, but with a slightly increased trypanocidal activity (Table 4). In contrast, a NBPM containing a 3-methoxy group' (LH18) was not metabolised by TbNTR and did not kill trypanosomes in the concentration range tested. Incorporation of electron-withdrawing groups such as fluoro (LH32), trifluoromethyl (LH33) and chloro (LH34) at the 2-position dramatically increased TbNTR activity and considerably improved the trypanocidal properties. LH32 and 33 have IC₅₀ values of 268±8 nM and 149±20 nM respectively, while LH34 has an IC₅₀ value of 8±1 nM. The latter value is 225-fold lower than that calculated for nifurtimox. To evaluate whether the increase in enzymatic and trypanocidal activity was specifically due to halogenation at the 2-position, a 3-fluorinated nitrobenzyl compound was examined (LH31). This molecule was efficiently metabolised by TbNTR, but had a parasite killing activity on par with LH7 (Tables 3 and 4). Therefore, although halogenation at any position on the phenyl ring is sufficient to stimulate TbNTR activity, only 2halogenated compounds have increased trypanocidal activity. Analysis of a difluorinated (2,6difluororo) compound (LH37) showed that it had the highest ThNTR activity and lowest IC50 value of any of the NBPMs tested. To summarize, inventors have determined that the optimal NTRactivated structures are acyclic 2-halogenated 4-nitrobenzyl phosphoramides which can be additionally halogenated in their 6-position (FIG. 3A). The compounds were also tested against intracellular form T. cruzi, insect form Leishmania major, and mammalian Vero cells (Tables 5 and 6).

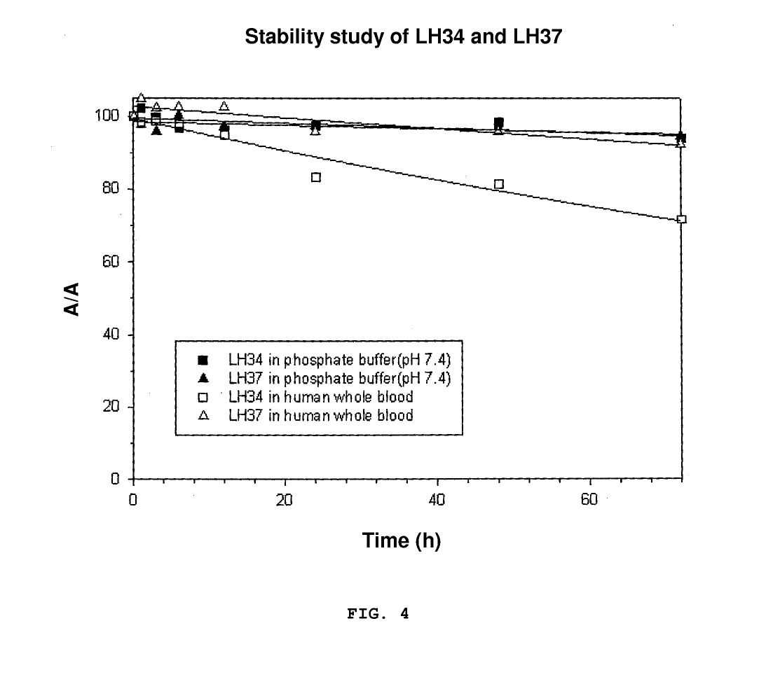

Stability studies indicate that LH37 is completely stable in pH 7.4, 100 mM phosphate buffer and human whole blood over a three-day incubation period at 37° C. while LH34 is stable in pH 7.4, 100 mM phosphate buffer and has less than 30% change in peak area in whole human blood under the same conditions (FIG. 4).

One possible explanation for the increased enzymatic and trypanocidal activity of the halogenated NBPMs could be the electronic inductive and resonance properties displayed by chlorine and fluorine. Prior to NTR mediated activation, the phenyl ring contains two electron-withdrawing substituents, a nitro group and a halogen. The electron-withdrawing inductive effect of the halogen increases the potential of the nitro group being reduced by NTR. NTR reduction converts the electron-withdrawing nitro group to an electron-donating hydroxylamine. This pushes electrons on the aromatic ring to the para benzylic carbon promoting cleavage of the benzylic C—O bonding and the release of the phosphoramide mustard. This cleavage is further facilitated by the electron-donating resonance effect of halogen substitution at the 2-position. The combined electronic effect of hydroxylamine at the 4-position and halogen at the 2-position causes a faster flux of electrons around the aromatic ring through to the benzylic C—O bond. This should increase the rate at which the compound fragments thus releasing the cytotoxic products.

The NBPMs metabolised in vitro by TbNTR are generally potent trypanocidal agents. To conclusively demonstrate this correlation, the susceptibility of T. brucei cell lines with reduced or elevated levels of NTR towards the most effective compounds (LH32, 33, 34 and 37) was investigated (FIGS. 3B and C) (34). In this context, trypanosomes with lower levels of NTR displayed relative resistance to all 4 compounds, whereas cells over expressing the enzyme exhibited hypersensitivity. This is in agreement with the phenotype shown by these parasite cell lines to other nitroheterocyclic compounds (34). As mammalian cells lack type I NTR, they should be less susceptible to agents that rely on this mechanism of activation. When we compared the relative toxicities of the most potent NBPMs against T. brucei and the THP-1 mammalian line, we observed that the 2 most effective trypanocidal agents were 1250-fold (LH34) and 986fold (LH37) more toxic to the parasite (Table 4). This difference is greater than that exhibited by nifurtimox (36-fold), a drug which is a key component of the recently sanctioned HAT treatment, NECT. However, it is important to stress that the in vitro toxicity data generated here is only against one mammalian cell line.

The following references are herein incorporated by reference in their entirety for all purposes:
1. Adagu, I. S., D. Nolder, D. C. Warhurst, and J. F. Rossignol. 2002. In vitro activity of nitazoxanide and related compounds against isolates of Giardia intestinalis, Entamoeba histolytica and Trichomonas vaginalis. J. Antimicrob. Chemother. 49:103-111.
2. Arya, S. C., and N. Agarwal. 2009. Nitrofurantoin: the return of an old friend in the wake of growing resistance. BJU Int. 103:994-995.
3. Baliani, A., G. J. Bueno, M. L. Stewart, V. Yardley, R. Bun, M. P. Barrett, and I. H. Gilbert. 2005. Design and synthesis of a series of melamine-based nitroheterocycles with activity against Trypanosomatid parasites. J. Med. Chem. 48:5570-5579.
4. Barrett, M. P., R. J. Burchmore, A. Stich, J. 0. Lazzari, A. C. Frasch, J. J. Cazzulo, and S. Krishna. 2003. The trypanosomiases. Lancet 362:1469-1480.
5. Checchi, F., P. Piola, H. Ayikoru, F. Thomas, D. Legros, and G. Priotto. 2007. Nifurtimox plus Efomithine for Late-Stage Sleeping Sickness in Uganda: A Case Series. PLoS Negl. Trop. Dis. 1:e64.
6. Chen, Y., and L. Hu. 2009. Design of anticancer prodrugs for reductive activation. Med. Res. Rev. 29:29-64.
7. Denny, W. A. 2003. Prodrugs for Gene-Directed Enzyme-Prodrug Therapy (Suicide Gene Therapy). J. Biomed. Biotechnol. 2003:48-70.
8. Docampo, R., R. P. Mason, C. Mottley, and R. P. Muniz. 1981. Generation of free radicals induced by nifurtimox in mammalian tissues. J. Biol. Chem. 256:10930-10933.
9. Dong, Q., D. Barsky, M. E. Colvin, C. F. Maus, S. M. Ludeman, J. F. Moravek, 0. M. Colvin, D. D. Bigner, P. Modrich, and H. S. Friedman. 1995. A structural basis for a phosphoramide mustard-induced DNA interstrand cross-link at 5′-d(GAC). Proc. Natl. Acad. Sci. USA 92:12170-12174.
10. Erickson, L. C., L. M. Ramonas, D. S. Zaharko, and K. W. Kohn. 1980. Cytotoxicity and DNA cross-linking activity of 4-sulfidocyclophosphamides in mouse leukemia cells in vitro. Cancer Res. 40:4216-4220.
11. Grunberg, E., and E. H. Titsworth. 1973. Chemotherapeutic properties of heterocyclic compounds: monocyclic compounds with five-membered rings. Annu. Rev. Microbiol. 27:317-346.
12. Hay, M. P., R. F. Anderson, D. M. Ferry, W. R. Wilson, and W. A. Denny. 2003. Synthesis and evaluation of nitroheterocyclic carbamate prodrugs for use with nitroreductase-mediated gene-directed enzyme prodrug therapy. J. Med. Chem. 46:55335545.
13. Hiraku, Y., A. Sekine, H. Nabeshi, K. Midorikawa, M. Murata, Y. Kumagai, and S. Kawanishi. 2004. Mechanism of carcinogenesis induced by a veterinary antimicrobial drug, nitrofurazone, via oxidative DNA damage and cell proliferation. Cancer Lett. 215:141-150.
14. Hirumi, H., and K Hirumi. 1989. Continuous cultivation of Trypanosoma brucei blood stream forms in a medium containing a low concentration of serum protein without feeder cell layers. J. Parasitol. 75:985-989.
15. Hu, L., C. Yu, Y. Jiang, J. Han, Z. Li, P. Browne, P. R. Race, R. J. Knox, P. F. Searle, and E. I. Hyde. 2003. Nitroaryl phosphoramides as novel prodrugs for E. coli nitroreductase activation in enzyme prodrug therapy. J. Med. Chem. 46:4818-4821.
16. Jiang, Y., J. Han, C. Yu, S. O. Vass, P. F. Searle, P. Browne, R. J. Knox, and L. Hu. 2006. Design, synthesis, and biological evaluation of cyclic and acyclic nitrobenzyl phosphoramide mustards for E. coli nitroreductase activation. J. Med. Chem. 49:4333-4343.
17. Jiang, Y., and L. Hu. 2008. N-(2,2-Dimethyl-2-(2-nitrophenyl)acetyl)-4-aminocyclophosphamide as a potential bioreductively activated prodrug of phosphoramide mustard. Bioorg. Med. Chem. Lett. 18:4059-4063.
18. Kashanian, J., P. Hakimian, M. Blute, Jr., J. Wong, H. Khanna, G. Wise, and R. Shabsigh. 2008. Nitrofurantoin: the return of an old friend in the wake of growing resistance. BJU Int. 102:1634-1637.
19. Li, Z., J. Han, Y. Jiang, P. Browne, R. J. Knox, and L. Hu. 2003. Nitrobenzocyclophosphamides as potential prodrugs for bioreductive activation: synthesis, stability, enzymatic reduction, and antiproliferative activity in cell culture. Bioorg. Med. Chem. 11:4171-4178.
20. McCalla, D. R., C. Kaiser, and M. H. Green. 1978. Genetics of nitrofurazone resistance in Escherichia coli. J. Bacteriol. 133:10-16.
21. McCalla, D. R., A. Reuvers, and C. Kaiser. 1971. Breakage of bacterial DNA by nitrofuran derivatives. Cancer Res. 31:2184-2188.
22. Moreno, S, N., R. P. Mason, and R. Docampo. 1984. Reduction of nifurtimox and nitrofurantoin to free radical metabolites by rat liver mitochondria. Evidence of an outer membrane-located nitroreductase. J. Biol. Chem. 259:6298-6305.
23. Muller, J., J. Wastling, S. Sanderson, N. Muller, and A. Hemphill. 2007. A novel Giardia lamblia nitroreductase, G1NR1, interacts with nitazoxanide and other thiazolides. Antimicrob. Agents Chemother. 51:1979-1986.
24. Peterson, F. J., R. P. Mason, J. Hovsepian, and J. L. Holtzman. 1979. Oxygen-sensitive and -insensitive nitroreduction by Escherichia coli and rat hepatic microsomes. J. Biol. Chem. 254:4009-4014.
25. Priotto, G., C. Fogg, M. Balasegaram, O. Erphas, A. Louga, F. Checchi, S. Ghabri, and P. Piola. 2006. Three drug combinations for late-stage Trypanosoma brucei gambiense sleeping sickness: a randomized clinical trial in Uganda. PLoS Clin. Trials 1:e39.
26. Roldan, M. D., E. Perez-Reinado, F. Castillo, and C. Moreno-Vivian. 2008. Reduction of polynitroaromatic compounds: the bacterial nitroreductases. FEMS Microbiol. Rev. 32:474-500.
27. Searle, P. F., M. J. Chen, L. Hu, P. R. Race, A. L. Lovering, J. I. Grove, C. Guise, M. Jaberipour, N. D. James, V. Mautner, L. S. Young, D. J. Kerr, A. Mountain, S. A. White, and E. I. Hyde. 2004. Nitroreductase: a prodrug-activating enzyme for cancer gene therapy. Clin. Exp. Pharmacol. Physiol. 31:811-816.
28. Stewart, M. L., G. J. Bueno, A. Ballani, B. Klenke, R. Brun, J. M. Brock, I. H. Gilbert, and M. P. Barrett. 2004. Trypanocidal activity of melamine-based nitroheterocycles. Antimicrob. Agents Chemother. 48:1733-1738.
29. Stover, C. K., P. Warrener, D. R. VanDevanter, D. R. Sherman, T. M. Arain, M. H. Langhorne, S. W. Anderson, J. A. Towell, Y. Yuan, D. N. McMurray, B. N. Kreiswirth, C. E. Barry, and W. R. Baker. 2000. A small-molecule nitroimidazopyran drug candidate for the treatment of tuberculosis. Nature 405:962-966.
30. Streeter, A. J., and B. A. Hoener. 1988. Evidence for the involvement of a nitrenium ion in the covalent binding of nitrofurazone to DNA. Pharm. Res. 5:434-436.
31. Takahashi, M., S. lizuka, T. Watanabe, M. Yoshida, J. Ando, K. Wakabayashi, and A. Maekawa. 2000. Possible mechanisms underlying mammary carcinogenesis in female Wistar rats by nitrofurazone. Cancer Lett. 156:177-184.
32. Wei, M. X., T. Tamiya, R. J. Rhee, X. O. Breakefield, and E. A. Chiocca. 1995. Diffusible cytotoxic metabolites contribute to the in vitro bystander effect associated with the cyclophosphamide/cytochrome P450 2B 1 cancer gene therapy paradigm. Clin. Cancer. Res. 1:1171-1177.
33. Whiteway, J., P. Koziarz, J. Veall, N. Sandhu, P. Kumar, B. Hoecher, and I. B. Lambert. 1998. Oxygen-insensitive nitroreductases: analysis of the roles of nfsA and nfsB in development of resistance to 5-nitrofuran derivatives in Escherichia coli. J. Bacteriol. 180:5529-5539.
34. Wilkinson, S. R., M. C. Taylor, D. Horn, J. M. Kelly, and I. Cheeseman. 2008. A mechanism for cross-resistance to nifurtimox and benznidazole in trypanosomes. Proc. Natl. Acad. Sci. USA 105:5022-5027.
35. Zenno, S., H. Koike, A. N. Kumar, R. Jayaraman, M. Tanokura, and K. Saigo. 1996. Biochemical characterization of NfsA, the Escherichia coli major nitroreductase exhibiting a high amino acid sequence homology to Frp, a Vibrio harveyi flavin oxidoreductase. J. Bacteriol. 178:4508-4514.

It is feasible to extend the approaches described in the present application to develop other trypanocidal agents possibly by tagging different cytotoxic ligands onto various nitroaromatic rings. The ligands selected could target any biochemical pathways within the parasite. Therefore, in view of the present disclosure, as the type I NTR activation system is absent from the mammalian host and a number of trypanosome specific systems have already been characterised it may be possible to combine these features to develop new, safer and cheap treatments against these debilitating diseases. The specification and drawings are accordingly to be regarded in an illustrative manner rather than a restrictive sense.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A compound of formula (I): 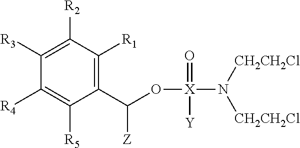
wherein:
R₂ is F;
R₃ is NO₂;
X is P;
Y is NH₂; and
R₁=R₄=R₅=Z=H.

2. The compound of claim 1, wherein:
R₁ is F;
R₃ is NO₂;
X is P;
Y is NH₂; and
R₂=R₄=R₅=Z=H.

3. The compound of claim 1, wherein:
R₁ is CF₃;
R₃ is NO₂;
X is P;
Y is NH₂; and
R₂=R₄=R₅=Z=H.

4. The compound of claim 1, wherein:
R₁ is Cl;
R₃ is NO₂;
X is P;
Y is NH₂; and
R₂=R₄=R₅=Z=H.

5. The compound of claim 1, wherein:
R₁=R₅=F;
R₃ is NO₂;
X is P;
Y is NH₂;
R₃=NO₂ and
R₂=R₄=Z=H.

6. The compound of claim 1, wherein:
R₁=R₂=R₄=R₅=F;
R₃ is NO₂;
X is P;
Y is NH₂;
R₃=NO₂ and
Z=H.

7. The compound of claim 1, wherein:
R₁=R₄=R₅=F;
R₃ is NO₂;
X is P;
R₂=Y is NH₂; and
R₃=NO₂ and
Z=H.

8. A pharmaceutical composition comprising a pharmaceutically acceptable excipient and an effective amount of the compound of claims 1-7.

9. The pharmaceutical composition of claim 8, wherein the compound is in an effective amount to treat an infection caused by Trypanosoma brucei or Trypanosoma cruzi.

10. A method for treating a protozoan infection, the method comprising administering a nitrobenzyl phosphoramide mustard to a subject infected with Trypanosoma brucei, Trypanosoma cruzi or other protozoan.

11. The method according to claim 10, wherein the nitrobenzyl phosphoramide mustard is a compound of formula (I): 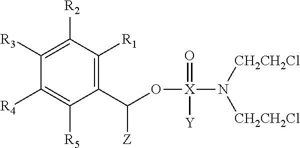
wherein:
R₁ is selected from the group consisting of hydrogen, a halogen and a halogenated C₁-C₄ alkyl;
R₂ is selected from the group consisting of hydrogen, a halogen, an amino, and a halogenated C₁-C₄ alkyl;
R₃ is NO₂;
R₄ is selected from the group consisting of hydrogen, a halogen, an amino, NO₂, a C₁-C₄ alkyl, and a C₁-C₄ methoxyalkyl;
R₅ is selected from the group consisting of hydrogen, a halogen, an amino, NO₂, a C₁-C₄ methoxyalkyl;
Z is selected from the group consisting of hydrogen, a C₁-C₄ alkyl and a C₁-C₄ methoxyalkyl;
X is selected from the group consisting of P, C, or S; and
Y is NH₂ or an aminoalkyl having 1-4 carbons; or
Y and Z form a ring structure and consist of —CH₂—CH₂—NH—.

12. The method according to claim 11, wherein R₁ is selected from the group consisting of hydrogen, Cl, F, Br, and CF₃; R₂ is hydrogen; R₃ is NO₂; R₄ is hydrogen; R₅ is hydrogen or Cl; Z is hydrogen; X is P or C; and Y is NH₂.

13. The method according to claim 11, wherein R₂ is not NO₂.

14. The method according to claim 11, wherein R₅ is —OCH₃.

15. The method according to claim 11, wherein R₁ is selected from the group consisting of Cl, Br, or F.

16. The method according to claim 11, wherein R1 and R5 are selected from the group consisting of Cl, Br, or F.

17. The method according to claim 11, wherein the nitrobenzyl phosphoramide mustard is administered as a pharmaceutical composition comprising a pharmaceutically acceptable excipient and a therapeutically effective amount of the compound of formula (I): 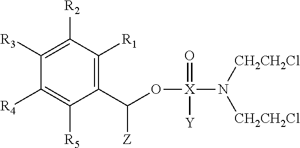
wherein:
R₁ is F;
R₃ is NO₂;
X is P;
Y is NH₂; and
R₂=R₄=R₅=Z=H.

18. The method according to claim 10, wherein the nitrobenzyl phosphoramide mustard is a compound of formula (II): 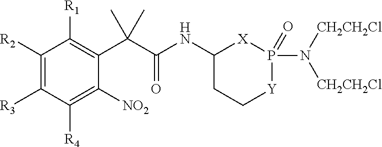
wherein:
X is O; and
Y is NH
R₁, R2, R3 and R₄ are each independently selected from the group consisting of hydrogen, a halogen and a halogenated C₁-C₄ alkyl.
